(19) United States
(12) Patent Application Publication
(10) Pub. No.: US 2008/0255489 A1
(43) Pub. Date: Oct. 16, 2008

(54) Knee-Ankle-Foot Orthosis with Load Brake

(75) Inventors: Eiichi Genda, Aichi-ken (JP); Kazushige Ohta, Aichi-ken (JP); Mitsuhisa Suzuki, Aichi-ken (JP); Shigeru Otsuka, Aichi-ken (JP); Kouji Yamamoto, Fukuoka-ken (JP)

Correspondence Address: OBLON, SPIVAK, MCCLELLAND MAIER & NEUSTADT, P.C., 1940 DUKE STREET, ALEXANDRIA, VA, 22314, US

(73) Assignees: Japan Labour Health and Welfare Organization, Kanagawa-Ken (JP); Imasen engineering Corporation, Aichi-ken (JP)

(21) Appl. No.: 12/089,213

(22) PCT Filed: Mar. 16, 2007
(86) PCT No.: PCT/JP2007/056134

§ 371 Date: Jun. 6, 2008

(30) Foreign Application Priority Data
Mar. 20, 2006 (JP) 2006-077661

(51) Int. Cl. A61F 5/00

(52) U.S. Cl. 602/27; 602/16; 602/26

(57) ABSTRACT

A knee-ankle-foot orthosis in accordance with the present invention can smoothly shift from the stance phase to the swing phase thereof. Namely, the knee-ankle-foot orthosis capable of securely locking a patient's knee in a desired angular position thereof with a load of his body weight, and immediately shifting his knee to the unlocked state without requiring any extra motion when the load disappears is provided.

As shown in FIG. 1, the speed of the rotation of a knee axis gear 23 adapted to transmit the rotation of a knee axis 21 is increased by eight times with a speed increasing gear train 25, and is transmitted to a brake drum 41. A wire 31 wound around the brake drum 41 is connected to first and second levers 27 and 28 as load detecting members. When a foot holding portion 18 contacts the ground and the first lever 27 is stepped down, the wire 31 is pulled downwardly with a load of his body weight, whereby brakes are applied. The brake torque is increased by eight times, and is transmitted to the knee axis 21. Consequently, even with a small force due to the load of his body weight, the knee-ankle-foot orthosis can be securely locked with a braking method to prevent giving way thereof. In addition, since the braking method is used, shifting to the unlocked state can be smoothly achieved.

### TECHNICAL FIELD

The present invention relates to a knee-ankle-foot orthosis adapted to provide independent walking means for a walking impaired person whose lower limb is paralyzed or whose muscle function is lost due to his spinal cord injury, cerebrovascular disorder, external wound etc.

### BACKGROUND ART

The knee-ankle-foot orthosis exhibits problems in locking and unlocking of a knee axis thereof. The most elementary knee-ankle-foot orthosis is provided with a device of locking and unlocking a thigh frame and a leg frame by hand, and upon walking, a patient locks the thigh frame and the leg frame, whereas, upon sitting, etc., he unlocks them to flex his knee. With the knee-ankle-foot orthosis thus arranged, however, a knee axis thereof is fixed upon walking so as to force him to walk with a locked knee. Consequently, his gait becomes unnatural, and he is forced to bear an increased burden. It is ideal for the knee-ankle-foot orthosis to be locked in the stance phase for supporting his body weight, and to be unlocked in the swing phase for allowing swinging of his leg. In one example of the knee-ankle-foot orthosis which is intended to realize such an ideal knee-ankle-foot orthosis, a knee axis is locked and unlocked with a claw.

FIG. 16 is a schematic diagram explaining an outline of a conventional knee-ankle-foot orthosis of which a knee axis is locked and unlocked with a claw. A circular plate 102 which rotates integrally with a thigh frame 101 has a diameter of which the size decreases in one part to define a step part 103. On the other hand, a claw 105 is pivotally attached to a leg frame with a pivot axis 106. A tip end of the claw 105 is connected to a heel of a foot holding portion 108 with a connecting rod 107. In the stance phase, as shown in FIG. 16(A), a patient's knee is extended so that the claw 105 contacts the step part 103 to stop and lock the rotation of the circular plate 102, thereby stopping the rotation of a knee axis 104. In the swing phase, as shown in FIG. 16(B), the claw 105 freely pivots so that the tip end of the claw 105 separates from the step part 103 and becomes free therefrom. Consequently, as shown in FIG. 16(C), the claw 105 does not engage with the step part 103 so that the circular plate 102 and the knee axis 104 turn to unlock his knee, whereby his knee can freely flex.

The locking-type knee-ankle-foot orthosis thus arranged, however, has the problem that the claw 105 bites the step part 103 to obstruct the patient's knee from smoothly shifting to its unlocked state in the swing phase. Namely, in the stance phase, a large load corresponding to a patient's body weight is applied between the tip end of the claw 105 and the step part 103 of the circular plate 102 so that the claw 105 bites the step part 103. As far as merely a small amount of a flexing moment is applied to his knee to apply a small pressing force between the claw 105 and the step part 103, the claw 105 does not separate from the step part 103 due to friction force to maintain the locked state even when the claw 105 becomes freely pivotable in the swing phase. This results in so-called biting of the claw 105 occurring. Accordingly, the patient has been required to further extend his knee for temporarily separating the claw 105 from the step part 103 and releasing the locked state. This is the phenomenon inevitably occurring when the locking method with claw is adopted, and requires a patient to make an extra motion at every step, which is troublesome.

By adopting not the locking method with claw but the braking method, the above-described biting phenomenon should be solved. Publication of unexamined Japanese patent application No. 2000-107212 discloses a prosthesis in which a load brake due to a patient's body weight is applied to a knee axis thereof. This is, however, a prosthesis which has a large space in a knee part thereof, and by providing a braking mechanism with a large volume therein, this prosthesis generates a large braking force which endures the patient's body weight. In the knee-ankle-foot orthosis, a braking mechanism must be provided in a small space beside the patient's knee so that it is impossible to adopt this method.

It has been considered that it is impossible for the knee-ankle-foot orthosis to apply brakes to a knee axis with a load due to a patient's body weight, because a braking force becomes short to cause giving way. In order to effect brakes which can securely prevent the giving way with the load due to the patient' body weight, a brake disc having a diameter of 30 cm or more is required for enduring a great torque, which is not practical. Publication of registered Japanese Utility Model No. 3002320 discloses a knee-ankle-foot orthosis with a braking mechanism. This knee-ankle-foot orthosis is, however, arranged that brakes are operated with power of a motor, but not applied with the patient's body weight. This orthosis also exhibits the problem that since brakes are operated with the motor, some time is required until brakes work, thereby generating a time lag so as to be inconvenient to use.

### DISCLOSURE OF THE INVENTION

As described above, the locking method with claw has the advantage that a knee axis can be securely locked, but has the problem that an extra motion is needed to shift it to the unlocked state because of the biting phenomenon of the claw. In addition, in the case of locking with a claw, the locking position is always limited to a constant knee position, normally its extended position. When the patient ascends stairs and slopes, his knee is needed to be locked in its slightly flexed position, but, with this method, his knee is locked only in its extended position so that there is exhibited the problem that he cannot ascend stairs, etc. Furthermore, the simple braking method has the problem that a braking force for the knee axis due to merely the load of the patient's body weight becomes short, and consequently, in order to generate a sufficient braking force, the diameter of a brake disc becomes excessively large so as not to be used practically.

The present invention has been made to solve the above-described problems, and has an object of providing a knee-ankle-foot orthosis which is convenient to use, is capable of securely locking a patient's knee in any angular position with a load due to his body weight by means of a braking device with a practical size without generating giving way, and is capable of shifting his knee to the unlocked state immediately when the load disappears without requiring any extra motion.

In order to achieve the above-described object, a first aspect of the knee-ankle-foot orthosis with load brake in accordance with the present invention is characterized in that the orthosis includes a knee axis gear which is secured to a thigh frame for taking a relative rotation of a knee axis to a leg frame, a speed increasing gear train which is rotatably supported with the leg frame for increasing the rotation speed of the knee axis gear, a braking section which is secured to a final stage of the speed increasing gear train for rotating integrally with a final stage gear, a load detecting section which is provided in a foot holding portion for detecting a contacting pressure of a foot, and braking means for connecting the load detecting section and the braking section to each other, and braking a rotation of the braking section when a load is applied to the load detecting section.

And in accordance with a second aspect of the invention, the orthosis is characterized by including a knee axis gear which is secured to a thigh frame for taking a relative rotation of a knee axis to a leg frame, a speed increasing gear train which is rotatably supported with the leg frame for increasing the rotation speed of the knee axis gear, a one-way clutch which is secured to a final stage of the speed increasing gear train for rotating integrally with a final stage gear, the one-way clutch being arranged such that an input stage is integral with the final gear, whereas a braking section is provided in an output stage, that when the leg frame rotates in a flexing direction thereof, the one-way clutch becomes on to stop the rotation with the braking section, and that when the leg frame rotates in an extending direction thereof, the one-way clutch becomes off to make the rotation thereof free, a load detecting section which is provided in a foot holding portion for detecting a contacting pressure of a foot, and braking means for connecting the load detecting section and the braking section to each other, and braking a rotation of the braking section when a load is applied to the load detecting section.

In this case, preferably, in accordance with a third aspect of the invention, the braking section is composed of a brake drum, the load detecting section is composed of a first lever which moves upwardly due to a load of a heel, and a second lever which has a portion adapted to move downwardly due to the load after reversing a motion of the first lever, and the braking means is composed of a wire, one end of which is secured to the second lever and the other end of which is secured to the leg frame after being wound around the brake drum.

In addition, in accordance with a fourth aspect of invention, the braking section is composed of a brake disc or a brake drum, the load detecting section is composed of an oil hydraulic transducer which detects a load of a heel and varies an oil pressure due to the load, and the braking means is composed of an oil hydraulic cylinder which is driven with the oil pressure of the oil hydraulic transducer, and one of a brake pad and a brake shoe, which is pressed with the oil hydraulic cylinder.

Furthermore, in accordance with a fifth aspect of the invention, the braking section is composed of a brake disc or a brake drum, the load detecting section is composed of a load detector which detects a load of a heel and converts the load to an electric signal, and the braking means is composed of command value calculating means for calculating a brake torque command value required corresponding to the electric signal from the load detector, an actuator which is driven with an output of the command value calculating means, and one of a brake pad and a brake shoe, which is driven and pressed with the actuator.

In addition, in accordance with a sixth aspect of the invention, the orthosis includes a planetary gear mechanism of which a first input axis is secured to a thigh frame, and a second input axis is secured to a leg frame for increasing a speed of a relative rotation of the first input axis and the second input axis, and transmitting the relative rotation to an output axis, a braking section to which a rotation of the output axis of the planetary gear mechanism is transmitted, a load detecting section which is provided in a foot holding portion for detecting a contacting pressure of a foot, and braking means for connecting the load detecting section and the braking section to each other, and braking a rotation of the braking section when a load is applied to the load detecting section.

Furthermore, in accordance with a seventh aspect of the invention, a one-way clutch is interposed between the output axis of the planetary gear mechanism and the braking section such that when the direction of a relative rotation of the thigh frame and the leg frame is the direction in which a knee flexes, the rotation is transmitted, and that when the direction of the relative rotation of the thigh frame and the leg frame is the direction in which the knee extends, the rotation is not transmitted.

And in accordance with an eighth aspect of the invention, the braking section is composed of a brake drum, the load detecting section is composed of a foot portion which includes an inner shell on which a foot of a human body is adapted to be put on, an outer shell which is adapted to contact a floor, and an elastic body which is interposed between the inner shell and the outer shell, and the braking means which is provided with a brake belt which is wound around the brake drum, one end of which is secured to a frame of the planetary gear mechanism, and the other end of which is connected to the inner shell of the foot portion.

With the arrangement of the first aspect of the invention, the speed of the relative rotation of the knee axis is increased and transmitted to the braking section. In other words, the braking torque of the braking section is increased and transmitted to the knee axis. Consequently, even when the radius of the braking section is small so that a merely small braking torque can be generated, a large braking torque is obtained in the knee axis after increased. Therefore, even when brakes are applied by a braking section with a small radius, which is easy to be attached to the knee-ankle-foot orthosis, a load torque due to the patient's body weight can be sufficiently supported to prevent the occurrence of giving way. In addition, since braking and locking are achieved with the braking mechanism, there is the operational advantage that upon releasing braking and locking, unpleasant phenomena due to biting of locking members such as a claw do not occur. And, since a large brake torque is not required for the braking section, there is the operational advantage that the construction around the braking section can be made simple.

When the hoot holding portion contacts the ground, and a load due to the patient's body weight is applied, the braking section is braked due to the load. Since the speed of the braking section is increased, the braking torque of the braking section is increased and then transmitted to the knee axis. Therefore, a sufficient braking torque can be transmitted to the knee axis only by the load due to the body weight so that the knee can be locked securely in a desired position. Since locking is achieved with the braking mechanism, upon unlocking, there occurs no bad effect such as biting phenomena of claws, whereby when the load on the foot holding portion is eliminated, the braking force immediately disappears. Consequently, there are exhibited the operational advantages that by merely bringing the foot holding portion into contact with the ground, the knee can be securely locked in a desired angular position with the load due to the patient's body weight, and that by merely separating the foot holding portion from the ground, the load disappears, and the knee can be immediately shifted to the unlocked state without any additional motion. Therefore, there is achieved the operational advantage that the knee-ankle-foot orthosis can smoothly shift from the stance phase to the swing phase, or from the swing phase to the stance phase, thereby improving a feeling upon using. And, since the orthosis can be locked in a desired angular position of the knee, stairs and slopes can be ascended, and standing-up motions can be facilitated.

In accordance with the second aspect of the invention, since the one-way clutch is provided between the final stage gear and the braking section, brakes can be applied only in the direction in which the knee flexes, whereby the knee can be extended freely in the direction in which the knee extends even when the load is applied to the foot portion. Therefore, there is exhibited the operational advantage that the knee-ankle-foot orthosis becomes convenient to use. For example, when a patient stands up, his knee must gradually extend from the flexed position with both lower limbs loaded. Since this mechanism is locked only in its flexing direction but is free in its extending direction, safe and smooth standing-up becomes possible. Upon ascending stairs, his foot contacts an upper stair with the knee-ankle-foot orthosis slightly flexed, thereby applying a load to lock his knee, and then, by kicking with a good foot, his body is lifted up. In the course of lifting his body, the knee of the orthosis gradually extends, and when the good foot contacts the ground, the knee of the orthosis becomes in approximately completely extended position. Even when the load applied to the knee-ankle-foot orthosis does not become completely 0, it can extend so as to be very easy to use.

In accordance with the third aspect of the invention, the braking section is composed of a brake drum, and the braking means is composed of a wire wound around the brake drum. With this arrangement, there is the operational advantage that the braking mechanism has a simple and lightweight construction. In addition, there is the operational advantage that by properly determining the number of winding of the wire around the brake drum, the brake torque can be properly determined.

In addition, in accordance with the fourth aspect of the invention, since the oil pressure is used, there is the operational advantage that the load of body weight can be transmitted to the brake without any loss, and that when the load disappears, the brake torque disappears with a quick response characteristic. Furthermore, since the oil pressure is used, there is achieved the operational advantage that the braking mechanism can be constructed freely.

In addition, in accordance with the fifth aspect of the invention, since brakes are controlled using an electric signal, there is the operational advantage that various controls are possible. By calculating and outputting a necessary and sufficient brake torque command value based on a detected heel load value, for example, at the time the heel load is weak, the current to be supplied to the actuator is reduced, whereas at the time the heal load is strong, such a brake torque command value as to overcome the same is generated, whereby the consumption of a battery can be restrained while ensuring locking of the knee. And, by detecting a toe load in addition to the heel load with the load detecting section, a brake torque command value is outputted while comparing the toe load and the heel load to each other, whereby a higher grade of knee joints control becomes possible.

In accordance with the sixth aspect of the invention, a planetary gear mechanism is used as the speed-increasing mechanism. This arrangement achieves the operational advantages that a lightweight and compact speed-increasing mechanism having a high speed increasing ratio can be constructed so as to be preferably attached to a side part of a knee axis of the knee-ankle-foot orthosis. In addition, there is the operational advantage that it is easy to balance the force applied to frames from an output axis of which the rotation speed is increased.

And in accordance with the seventh aspect of the invention, since the one-way clutch is interposed, similar operational advantages to those described in the second aspect of the invention are achieved.

Furthermore, in accordance with the eighth aspect of the invention, since the load applied to the foot portion is detected with the compression of an elastic body which is interposed between an inner shell and an outer shell, the load can be detected securely, and since no additional member is provided outside the outer shell, there does not exist any obstacle upon walking. In addition, since the braking means is composed of a brake belt which is wound around a brake drum, the brake drum can be tightened securely and brakes can be applied by virtue of the relative movement of the inner shell due to the load of the foot portion.

### BRIEF EXPLANATION OF THE DRAWINGS

FIG. 1 is a perspective view showing a first embodiment of a knee-ankle-foot orthosis with load brake in accordance with the present invention;

FIG. 2 is a side view showing a foot holding portion;

FIG. 3(A) is a sectional view showing a three-stage speed increasing gear train, and FIG. 3(B) is a front view showing the same.

FIG. 4 is a perspective view showing a knee axis control unit of a knee-ankle-foot orthosis in a second embodiment, in which mechanisms such as a brake drum, a speed increasing gear train, etc. are made into a unit.

FIG. 5 is a rear view showing the knee axis control unit of the knee-ankle-foot orthosis in the second embodiment.

FIG. 6 is a longitudinal sectional view of the knee axis control unit of the knee-ankle-foot orthosis in the second embodiment.

FIG. 7 is a perspective view showing a knee axis control unit of a knee-ankle-foot orthosis in a third embodiment, to which a one-way clutch is added to form a unit.

FIG. 8 is a longitudinal sectional view of the knee axis control unit of the knee-ankle-foot orthosis in the third embodiment, to which the one-way clutch is added.

FIG. 9 is a perspective view showing a fourth embodiment of a knee-ankle-foot orthosis with load brake, which uses an oil pressure.

FIG. 10 is a perspective view showing a fifth embodiment of a knee-ankle-foot orthosis with load brake, which uses an electric signal.

FIG. 11 is a perspective view showing a knee-ankle-foot orthosis which is provided with a knee axis control unit with a one-way clutch and an improved foot portion;

FIG. 12 is a side view showing the improved foot portion.

FIG. 13 is a perspective view showing a knee axis control unit which uses a planetary gear mechanism.

FIG. 14 is a diagram showing the planetary gear mechanism.

FIG. 15 is an exploded perspective view showing the knee axis control unit which uses the planetary gear mechanism.

FIG. 16 is a diagram explaining an outline of a conventional knee-ankle-foot orthosis of which a knee axis is locked and unlocked with a claw.

### BEST MODE FOR CARRYING OUT THE INVENTION

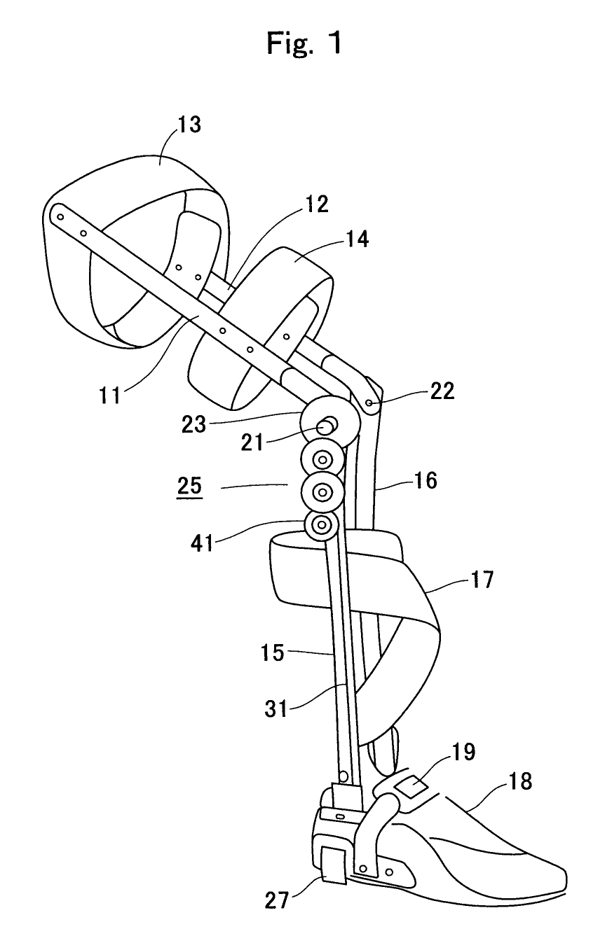

FIG. 1 is a perspective view showing a first embodiment of a knee-ankle-foot orthosis with load brake in accordance with the present invention. Thigh bands 13 and 14 are attached to two left and right thigh frames 11 and 12. Knee axes 21 and 22 are secured to lower ends of the thigh frames 11 and 12, and leg frames 15 and 16 are pivotally attached to the knee axes 21 and 22. A leg band 17 is attached to the leg frames 15 and 16. Afoot holding portion 18 is secured to lower ends of the leg frames 15 and 16, and a foot band 19 is attached to the foot holding portion 18. A knee axis gear 23 is secured to the knee axis 21 located on the inner side of the orthosis, and the knee axis gear 23 engages with a three-stage speed increasing gear train 25 which is rotatably supported on the leg frame 15.

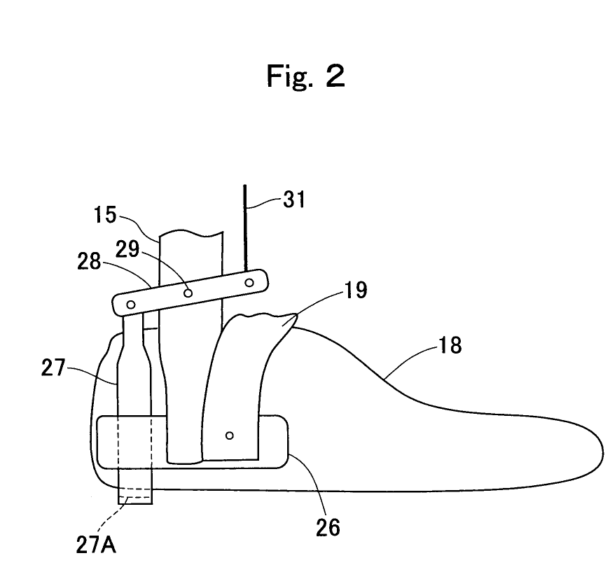

FIG. 2 is a side view showing the foot holding portion 18. A side plate 26 is secured to a side part of the foot holding portion 18 to support the leg frame 15. A first lever 27 is held on a rear side of the side plate 26 so as to slide upwardly and downwardly. A lower part of the first lever 27 bends through 90 degrees, and a horizontal bending part 27A is located under a heel of the foot holding portion 18. An upper end of the first lever 27 is connected to a rear end of a second lever 28 with a pin. The second lever 28 is pivotally supported with the leg frame 15 via an axis 29 in a lengthwise center thereof. A lower end of a wire 31 which extends downwardly is connected to a front end of the second lever 28. When the heel contacts the ground, the bending part 27A of the first framer 27 contacts the ground to push the first frame 27 upwardly with a load of a patient' body weight. This motion is reversed with the second lever 28 to pull the wire 31 downwardly with the load of the patient's body weight. The first lever 27 and the second lever 28 define a load detecting section.

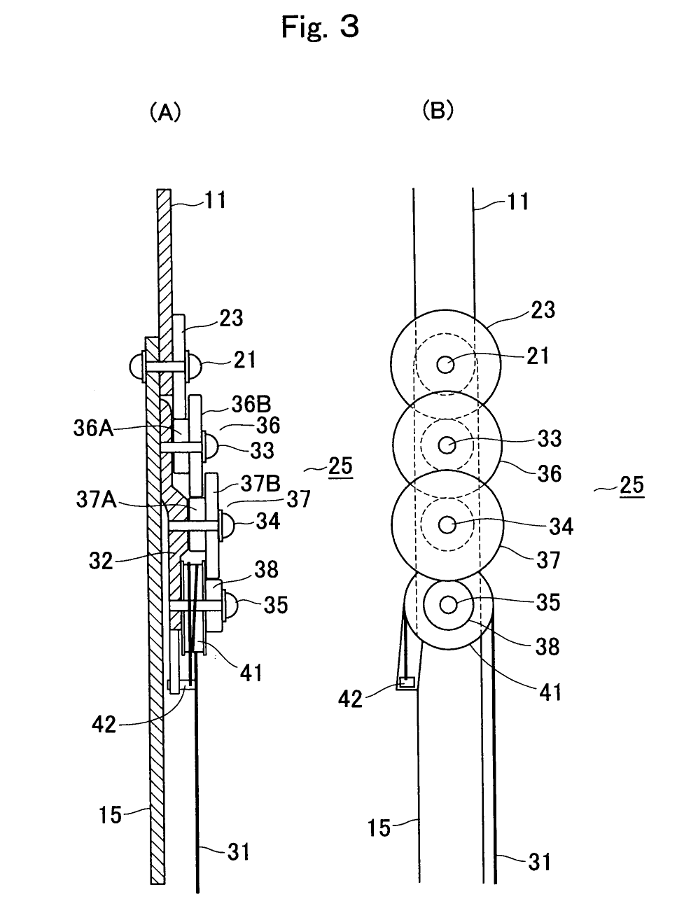

FIG. 3 is a sectional view (A) and a front view (B) showing the three-stage speed increasing gear train 25. The leg frame 15 is pivotally supported with the knee axis 21 which is secured to the thigh frame 11. And the knee axis gear 23 is secured to the thigh frame 11 and the knee axis 21. A support stay 32 is secured to the leg frame 15, and three axes 33, 34 and 35 are provided in the support stay 32. A first-stage speed increasing gear 36, a second-stage speed increasing gear 37, a third-stage (final stage) speed increasing gear 38 are rotatably supported with the first-stage axis 33, the second-stage axis 34, and the third-stage axis 35, respectively. The first-stage speed increasing gear 36 is consisting of a small diameter gear 36A and a large diameter gear 36B, and the small diameter gear 36A and the large diameter gear 36B are secured to each other so as to rotate integrally. Similarly, the second-stage speed increasing gear 37 is consisting of a small diameter gear 37A and a large diameter gear 37B, and the small diameter gear 37A and the large diameter gear 37B are secured to each other so as to rotate integrally. The third-stage speed increasing gear 38 is a small diameter gear. A brake drum 41 is secured to the third-stage speed increasing gear 38 so as to rotate integrally.

And, the knee axis gear with a large diameter 23 engages with the first-stage small diameter gear 36A, the first-stage large diameter gear 36B engages with the second-stage small diameter gear 37A, and the second-stage large diameter gear 37B engages with the third-stage small diameter gear 38. More specifically, gears, each having a pitch circle diameter of 36 mm and 24 teeth, were used as the large diameter gears 23, 36B and 37B, whereas gears, each having a pitch circle diameter of 18 mm and 12 teeth, were used as the small diameter gears 36A, 37A and 38. The face width thereof is 5 mm. The speed increasing ratio of the three-stage speed increasing gear train 25 becomes 8 times (2×2×2). And the diameter of the brake drum 41 was determined to 28 mm. The wire 31 was wound around this brake drum 41 by 3/2 times, and an end of the wire 31 is secured to an anchor 42 which is secured to the support stay 32.

The operation of the three-stage speed increasing gear thus arranged will be explained. In the stance phase, the knee-ankle-foot orthosis contacts the ground, and the patient' body weight is applied to the bending part 27A of the first lever 27. Consequently, the wire 31 is pulled downwardly due to the load of the body weight, and a tension force due to the load is generated in the wire 31. This tension force is also transmitted to the wire 31 wound around the brake drum 41 to tighten the brake drum 41, thereby generating a brake torque force in the brake drum 41 due to friction between the wire 31 and the brake drum 41. This brake torque force is increased with the speed increasing gear train 25 by eight times, and is transmitted to the knee axis gear 23. Therefore, with only the load of the body weight, the relative rotation of the knee axis gear 23, namely the thigh frame 11, can be securely prevented, and his knee can be locked. As a result, dangerous giving way in the stance phase can be prevented.

When the foot holding portion 18 of the knee-ankle-foot orthosis separates from the ground, the bending part 27A of the first lever 27 also separates from the ground, and the load which has been applied to the first lever 27 disappears. Consequently, the tension force of the wire 31 also disappears and the friction torque of the wire 31 and the brake drum 41 is eliminated, whereby the brake drum 41 can rotate freely. As a result, the relative rotation of the knee axis gear 23 becomes also free to unlock his knee. In this state, his knee can freely swing, and his leg can freely swing so as to be suited to the swing phase. Shifting from the stance phase in which his knee is locked to the swing phase in which his knee is unlocked can be smoothly achieved in a moment when the foot holding portion 18 merely separates from the ground, whereby the knee-ankle-foot orthosis can be used comfortably. In addition, even when the knee of the knee-ankle-foot orthosis is in the flexed state, the patient's knee is locked with the load of his body when the foot holding portion 18 contacts the ground, so that the knee-ankle-foot orthosis can be used in various situations. For example, it becomes possible to walk up stairs and slopes. Locking and unlocking of his knee can be changed over by meerly bringing the hoot holding portion 18 into contact with the ground or separating it therefrom without any extending motion of his knee so that the knee-ankle-foot orthosis can be used easily and comfortably.

And, mechanisms such as the brake drum 41, the speed increasing gear train 25, etc. are arranged in a space with 4 cm in width and 15 cm in length so as not to become bulky, and obstruct the movement of the patient. And it has been confirmed from calculation results and experimental orthoses that the brake torque caused by the friction force of the wire 31 and the brake drum 41 along with the three-stage speed increasing gear train 25 can produce a large brake torque enough to support the patient's body weight, and that each of these members can exhibit sufficient strength.

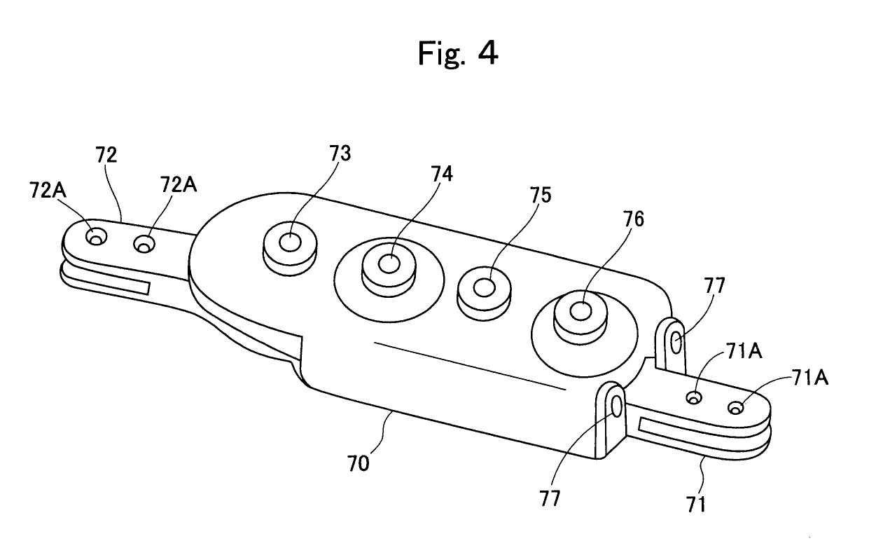

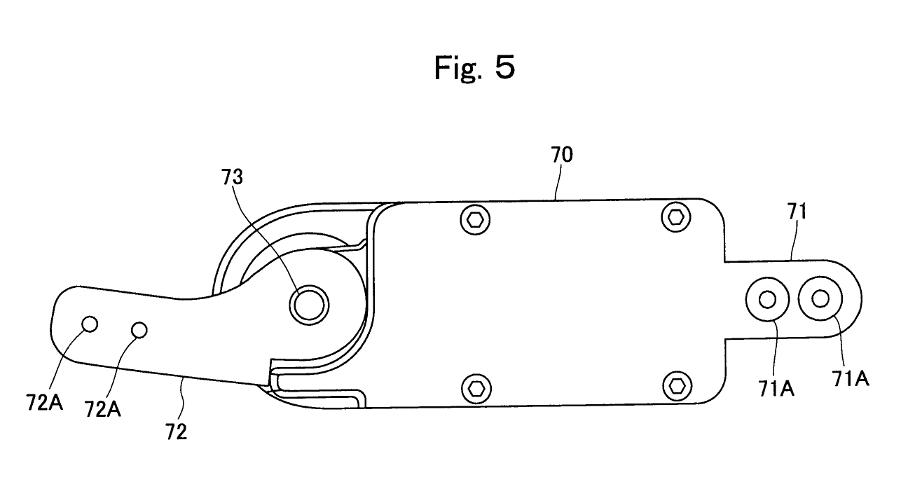

FIG. 4 is a perspective view showing a knee axis control unit of the knee-ankle-foot orthosis in a second embodiment, in which mechanisms such as the brake drum 41, the speed increasing gear train 25, etc. are formed into a unit. FIG. 5 is a rear view of the knee axis control unit. A unit housing 70 is secured to the leg frame 15, and is formed integrally with a leg arm 71. Two screw holes 71A and 71A are provided in the leg arm 71 for securing the unit housing 70 to the leg frame 15. On the opposite side of the unit housing 70, a thigh arm 72 is pivotally supported with the unit housing 70 via a knee axis 73. Two screw holes 72A and 72A are provided in the thigh arm 72 for securing the thigh arm 72 to the thigh frame 11. In addition to the knee axis 73, a first-stage axis 74, a second-stage axis 75 and a third-stage axis 76 are provided in the unit housing 70. Two wire holes 77 and 77 are provided in a side surface of the unit housing 70 on the side of the leg arm 71, and the wire 31 for braking is adapted to be inserted therein.

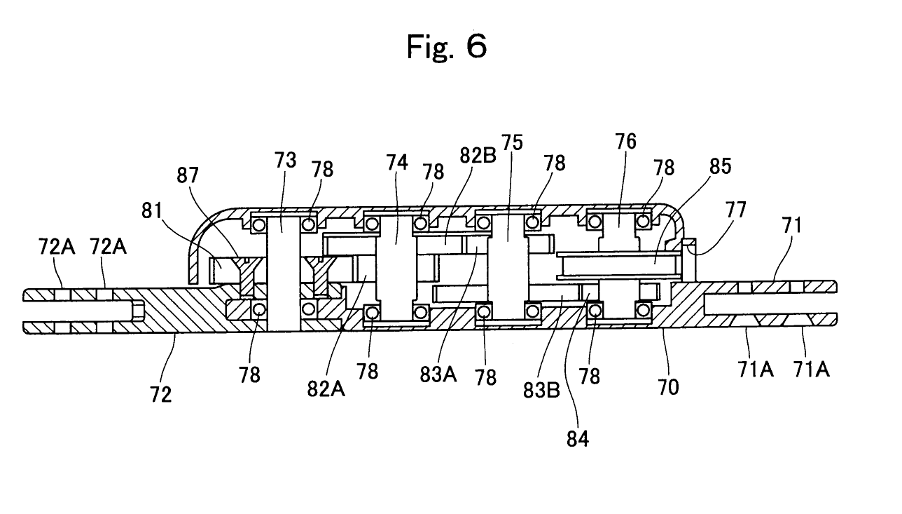

FIG. 6 is a longitudinal sectional view of the knee axis control unit. The axes 73, 74, 75 and 76 are rotatably supported with the unit housing 70 via bearings 78 and 78, respectively. The thigh arm 72 is secured to the knee axis 73 so as to rotate integrally therewith. A large diameter knee axis gear 81 is secured to the thigh arm 72 with screws 87. The knee axis gear 81 engages with a first-stage small diameter gear 82A, a first-stage large diameter gear 82B engages with a second-stage small diameter gear 83A, and a second-stage large diameter gear 83B engages with a third-stage small diameter gear 84. More specifically, gears, each having a pitch diameter of 36 mm and 24 teeth, were used as the large diameter gears 81, 82B and 83B, and gears, each having a pitch diameter of 18 mm and 12 teeth, were used as the small diameter gears 82A, 83A and 84. The face width thereof is 5 mm. The speed increasing ratio of the three-stage speed increasing gear train 25 becomes 8 times (2×2×2). Each of the gears 81, 82A, 82B, 83A, 83B and 84 is secured to the axis 73, 74, 75 or 76, and rotates integrally with the axis 73, 74, 75 or 76.

A brake drum 85 is secured to the final third-stage axis 76 to rotate integrally with the third-stage gear 84. The wire 31 which is connected to the second lever 28 (FIG. 2) of the foot holding portion 18 of the knee-ankle-foot orthosis is wound around this brake drum 85, and when a tension force is generated in the wire 31, a brake torque is generated in the brake drum 85. The brake torque of the brake drum 85 is increased by eight times, and is transmitted to the knee axis 73.

By forming the speed increasing gear train into a unit in this manner, there is achieved the operational advantage that it becomes easy to remodel the conventional knee-ankle-foot orthosis to the knee-ankle-foot orthosis with load brake by attaching this unit housing 70 thereto.

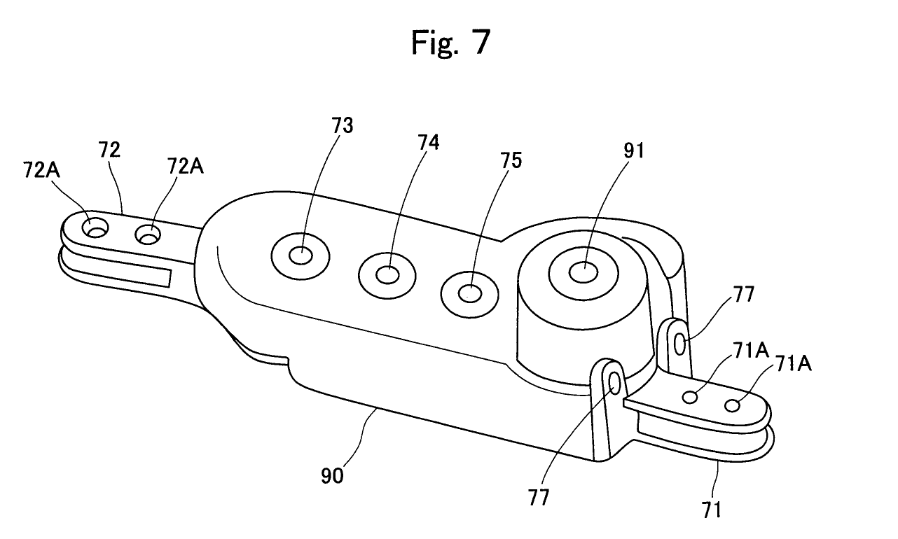

FIG. 7 is a perspective view showing a knee axis control unit of the knee-ankle-foot orthosis in a third embodiment, in which mechanisms including a one-way clutch are formed into a unit. A rear view is identical to FIG. 5 so as not to be specifically shown. Like reference characters will be used to designate members common to those shown in FIG. 4, and descriptions thereof will be omitted. In the present embodiment, the axial length of a final third-stage axis 91 is made slightly longer than those of the other axes 73, 74 and 75, and accordingly, a unit housing 90 is configured to have an increased wall thickness around the third-stage axis 91 for accommodating the one-way clutch.

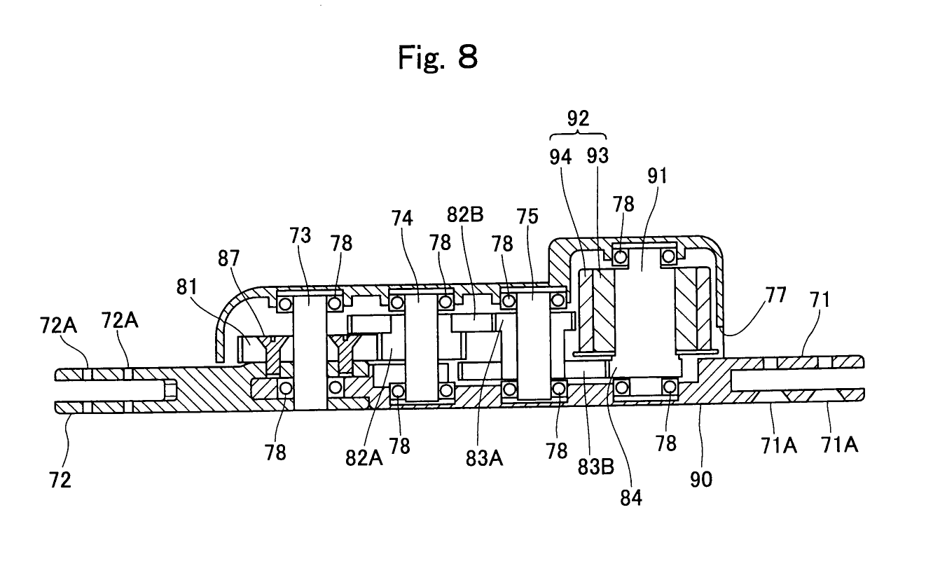

FIG. 8 is a longitudinal sectional view of a knee axis control unit, to which the one-way clutch is added. Like reference characters will be used to designate members common to those shown in FIG. 6, and descriptions thereof will be omitted. The final third-stage axis 91 is rotatably supported with the bearings 78 and 78 in the unit housing 90. A third-stage speed increasing gear 84 with a small diameter is formed in a lower part of the axis 91. The gear train is formed such that the speed increases from the knee axis gear 81 to the third-stage speed increasing gear 84 by eight times. A one-way clutch 92 is added around an upper part of the axis 91. The one-way clutch 92 is a clutch on the market, and includes an inner ring 93 and an outer ring 94, and the inner ring 93 is secured to the axis 91 to rotate integrally therewith. When the outer ring 94 rotates in one direction such as rightward, it rotates integrally with the inner ring 93, whereas when the outer ring 94 rotates in an opposite direction such as leftward, it separates from the inner ring 93 and freely rotates. The direction of the one-way clutch depends on the knee-ankle-foot orthosis being for use in a right lower limb or a left lower limb, and is arranged such that in the direction in which his knee flexes, clutch is applied, whereas in the direction in which his knee extends, the outer ring 94 freely rotates. And in the present embodiment, the wire 31 is wound directly around the outer ring 94 of the one-way clutch 92 so that the outer ring 94 also serves as the brake drum.

With this arrangement, brakes can be applied in only the direction in which his knee flexes so that his knee can freely extend even when a load is applied to the foot holding portion 18. Therefore, there is exhibited the operational advantage that the knee-ankle-foot orthosis can be used comfortably. For example, upon ascending stairs, a patient contacts an upper stair with the knee-ankle-foot orthosis slightly flexed, locks his knee by applying a load, and lifts his body with his good foot as a swing foot. When his body is lifted up and his good foot contacts the ground, the flexed knee-ankle-foot orthosis is intended to be extended. At this time, even when the load applied to the foot holding portion 18 of the knee-ankle-foot orthosis is not completely 0, the flexed knee-ankle-foot orthosis can be extended freely so as to be used very conveniently.

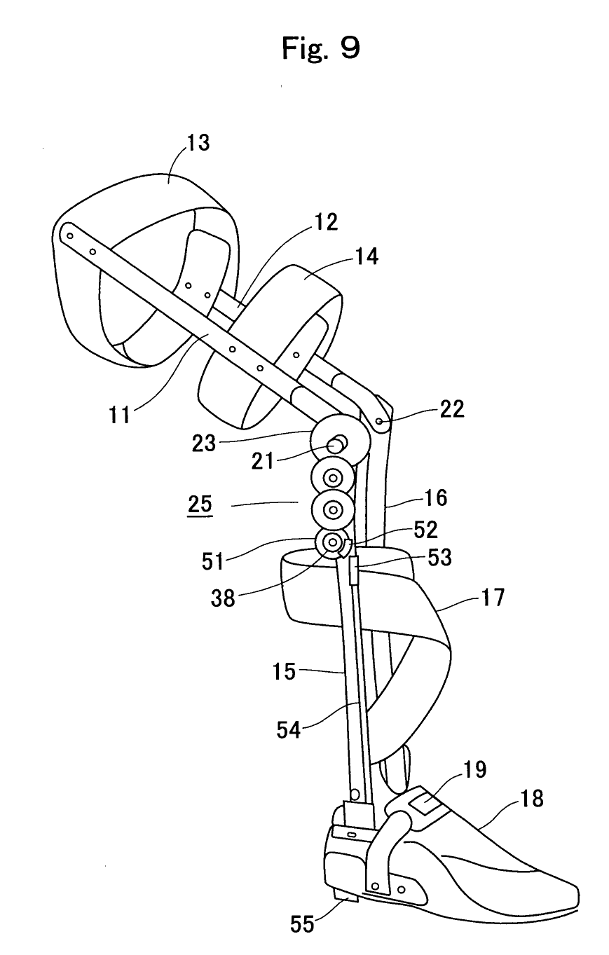

FIG. 9 is a perspective view showing a knee-ankle-foot orthosis with load brake in a fourth embodiment, which uses an oil pressure. Like reference characters will be used to designate members common to members such as the speed increasing gear train 25 disclosed in the first embodiment and shown in FIG. 1, and descriptions thereof will be omitted. A brake disc 51 is secured to the final-stage (third-stage) speed increasing gear 38 of the speed increasing gear train 25 so as to rotate integrally therewith. A brake pad 52 is arranged so as to hold the brake disc 51 from both surfaces thereof, and is supported with the leg frame 15. The brake pad 52 is arranged so as to be driven with an oil hydraulic cylinder 53. The oil hydraulic cylinder 53 is interconnected with an oil hydraulic transducer 55 of the foot holding portion 18 via an oil hydraulic pipe 54. The oil hydraulic transducer 55 is an oil chamber defined with a diaphragm or rubber, for example, and is provided in a bottom of a heel of the foot holding potion 18. The oil pressure transducer 55 detects the contacting pressure of the heel, and converts the same to an oil pressure, and the converted oil pressure is transmitted to the oil hydraulic cylinder 53 via the oil pressure pipe 54.

With the above-described arrangement, in the stance phase, the pressure of a heel load is converted to an oil pressure with the oil hydraulic transducer 55 to drive the oil hydraulic cylinder 53. Consequently, the brake pad 52 holds and presses the brake disc 51 to apply a brake torque to the brake disc 51. Even when the oil pressure is low and the bake torque applied to the brake disc 51 is small, the brake torque is increased with the speed increasing gear train 25 to eight times, and the increased brake torque is applied to the knee axis 21 so that the orthosis can securely endure the patient's body weight and lock the knee axis 21. In the swing phase, the heel separates from the ground so that the load which has been applied to the oil hydraulic transducer 55 disappears and the oil pressure becomes 0. Consequently, the brake pad 52 separates from the brake disc 51, and the knee axis 21 freely rotates and his knee freely swings. The above-described arrangement exhibits the operational advantages that the load due to the body weight can be transmitted to the brake without any loss, and that when the load disappears, the brake torque speedily disappears corresponding thereto. In addition, it exhibits the operational advantage that the braking mechanism is freely arranged. In the present embodiment, the braking mechanism is composed of a disc brake including the brake pad 52 and the brake disc 51, but alternatively, the braking mechanism can be composed of a drum brake including a brake drum and a brake shoe.

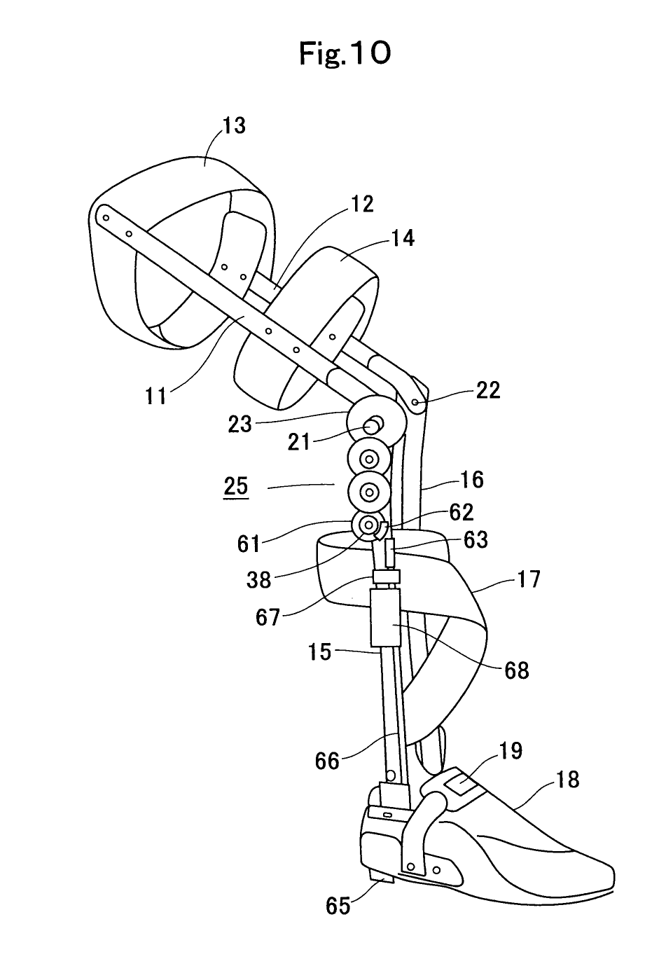

FIG. 10 is a perspective view showing a knee-ankle-foot orthosis with load brake in a fifth embodiment, which uses an electric signal. Like reference characters will be used to designate members common to members such as the speed increasing gear train 25 disclosed in the first embodiment and shown in FIG. 1, and descriptions thereof will be omitted. A brake disc 61 is secured to the final stage (third-stage) speed increasing gear 38 of the speed increasing gear train 25 so as to rotate integrally therewith. A brake pad 62 is arranged to hold the brake disc 61 from both surfaces thereof, and is supported with the leg frame 15. The brake pad 62 is arranged so as to be driven with an electromagnetic actuator 63. A load detector 65 composed of a strain gauge, etc. is attached to a bottom of a heel of the foot holding portion 18. An electric signal from the load detector 65 is transmitted to an electronic control unit 67 via a cable 66. Electric power is supplied to the electronic control unit 67 from a battery 68 which is held with the leg frame 15, and the electronic control unit 67 is arranged to drive the electromagnetic actuator 63 based on the electric signal from the load detector 65.

The electronic control unit 67 has an MPU (microprocessor unit) in an inside thereof, and controls a driving current of the electromagnetic actuator 63 into a nonlinear configuration according to a heel load detecting value from the load detector 65, thereby controlling the brake torque. The electronic control unit 67 defines brake torque command value calculating means. The brake torque which is necessary and sufficient for locking the knee axis 21 in the stance phase is commanded. The brake torque is reduced to ⅛ with the speed increasing gear train 25 so that there are exhibited the operational advantages that the electromagnetic actuator 63 may have a small power, and that the life of the battery 68 is extended. In addition, in the stance phase, the electric current to be applied to the electromagnetic actuator 63 is maintained to a predetermined saturation value if the heel load increases so that there is exhibited the operational advantage that the current consumption of the battery 68 decreases.

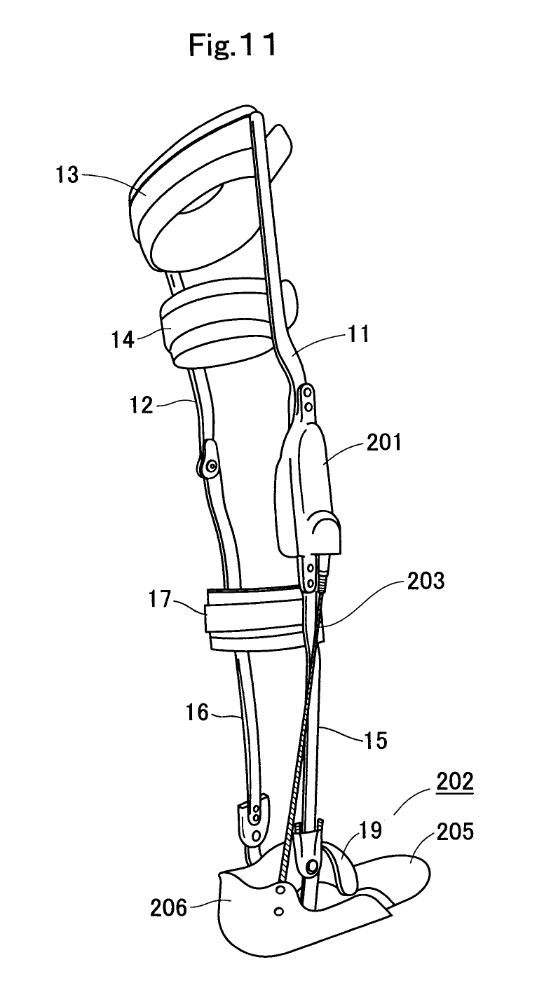

FIG. 11 is a perspective view showing a knee-ankle-foot orthosis to which a knee axis control unit with one-way clutch 201, and an improved foot portion 202 are added. A tube-shaped wire housing 203 extends from a lower end of the knee axis control unit 201 to the foot portion 202. The foot portion 202 has an inner plastic part (inner shell) 205 on which a foot of a human body is adapted to put, and an outer plastic part (outer shell) 206 which is adapted to contact a floor surface.

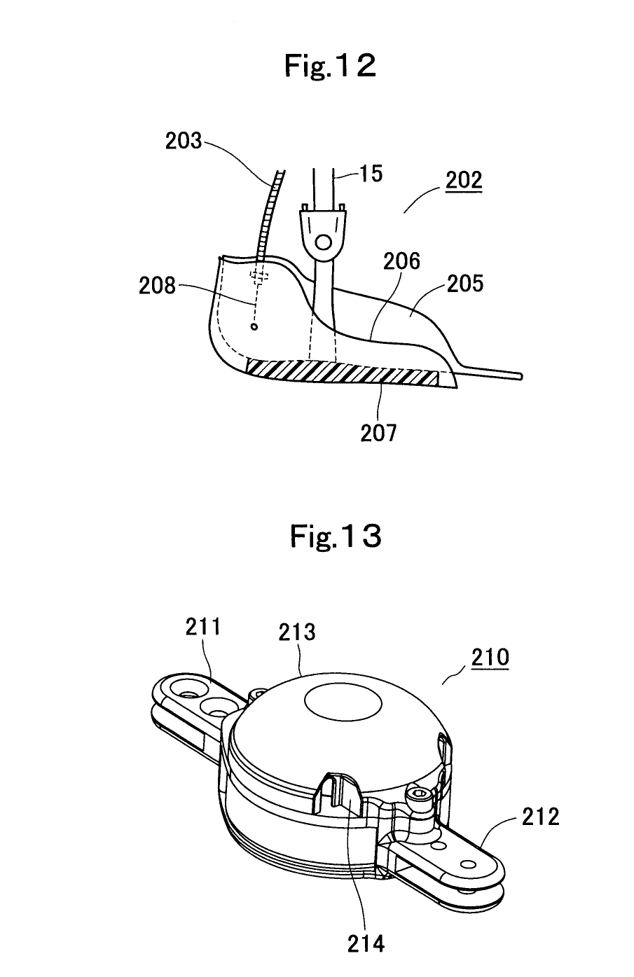

FIG. 12 is a side view showing an improved foot portion 202. A sponge-like elastic body 207 is provided in a foot bottom between the inner plastic part (inner shell) 205 and the outer plastic part (outer shell) 206 so as to be elastically compressed therebetween. The wire housing 203 extending from the knee axis control unit 201 is secured to the outer plastic part (outer shell) 206. A wire 208 inserted in the wire housing 203 is secured to the inner plastic part (inner shell) 205. When a load is applied to the foot portion 202, the elastic body 207 is compressed, and the inner plastic part 205 lowers. As a result, the wire 208 is pulled down to brake the knee axis control unit 201. When the load applied to the foot portion 202 disappears, the wire 208 comes loose due to the returning force of the elastic body 207, and the braking force for his knee disappears. The inner plastic part (inner shell) 205 on which the foot of the human body is adapted to put, the outer plastic part (outer shell) 206 which is adapted to contact the floor surface, and the elastic body 207 define a load detecting section.

FIG. 13 is a perspective view showing a knee axis control unit 210 which uses a planetary gear mechanism. A thigh arm 211 adapted to be secured to the thigh frame 11, and a leg arm 212 adapted to be secured to the leg frame 15 are provided therein. The thigh arm 211 and the leg arm 212 are arranged so as to rotate relative to each other. The planetary gear mechanism is assembled in a housing 213, and is provided with a portion adapted to increase the speed of the relative rotation of the thigh arm 211 and the leg arm 212. The portion which increases the speed is arranged to be braked with a belt. An outlet opening 214 for a brake belt is provided in a side part of the housing 213.

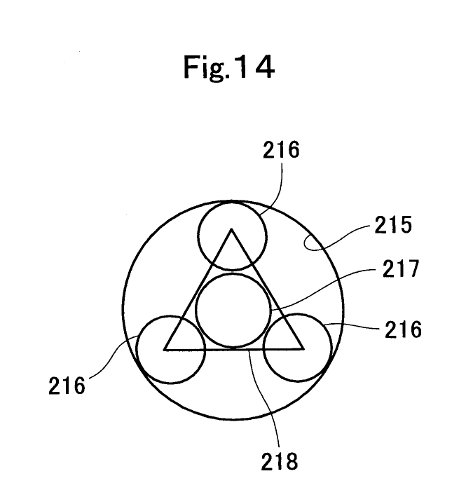

FIG. 14 is a diagram showing the planetary gear mechanism. This planetary gear mechanism is of 2S-C type. Three planetary gears 216 engage with a large diameter internal gear 215, and one small diameter sun gear 217 engages with the three planetary gears 216. The three planetary gears 216 are supported with a carrier 218. The thigh arm 211 is secured to the internal gear 215, and the leg arm 212 is secured to the carrier 218. The speed of the relative rotation of the internal gear 215 and the carrier 218 is increased to rotate the sun gear 217. The number of teeth (Za) of the internal gear 215 is 84, the number of teeth (Zb) of the sun gear 217 is 12, and the number of teeth (Zc) of the planetary gear 216 is 36. The speed increasing ratio of the sun gear 217 to the relative rotation of the internal gear 215 and the carrier 218 (1+Za/Zb) is 1+84/12, that is 8.

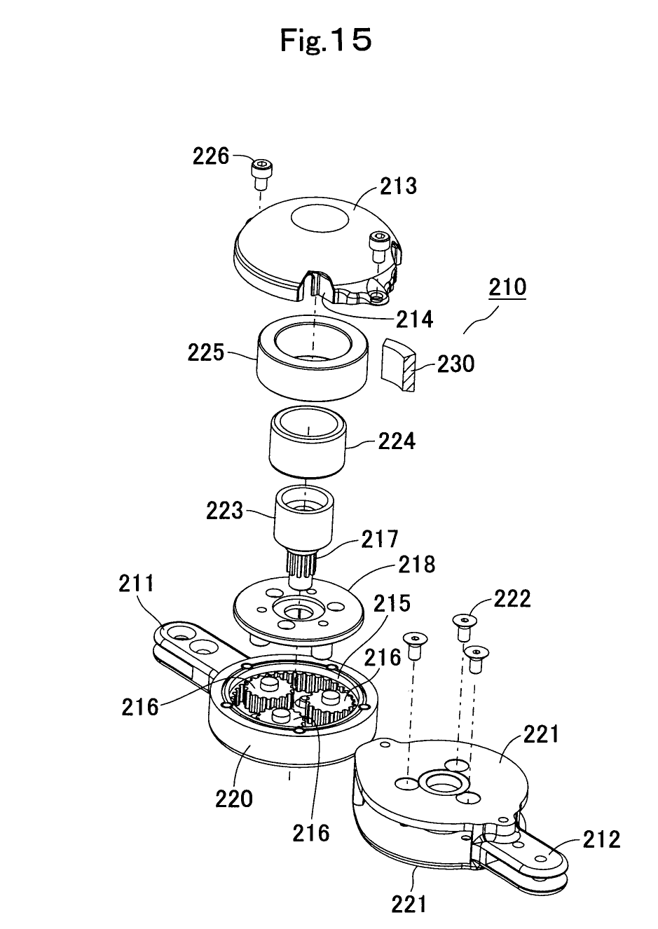

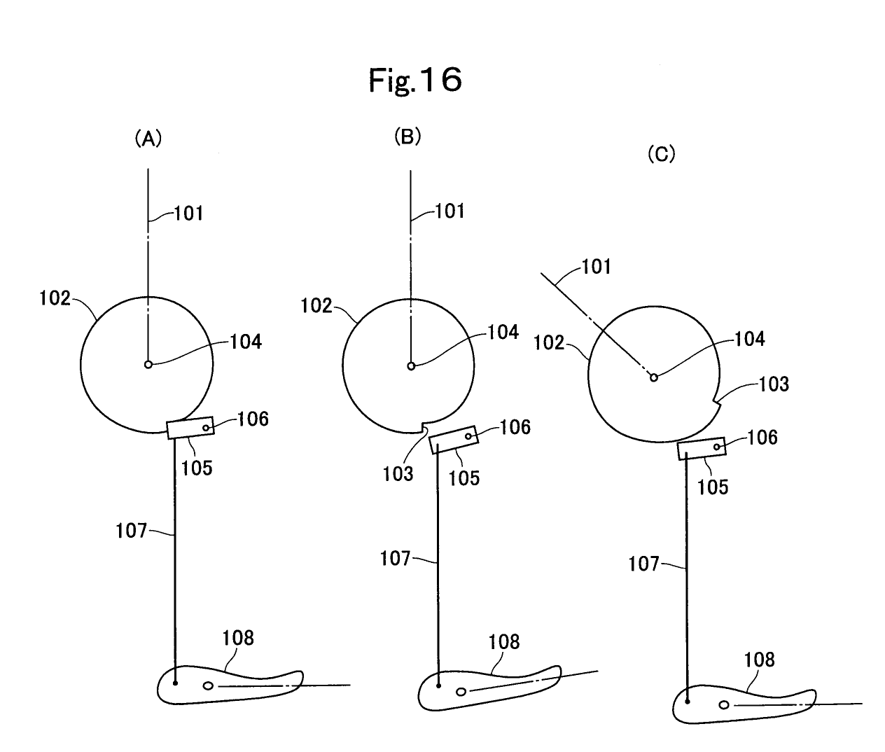

FIG. 15 is an exploded perspective view showing the knee axis control unit 210 which uses the planetary gear mechanism. The large diameter internal gear 215 is secured to an annular holder 220 which is integral with the thigh arm 211. On the other hand, the carrier 218 is secured to two sheets of upper and lower circular holders 221 which are integral with the leg arm 212 with bolts 222. The three planetary gears 216 are supported with the carrier 218. An output axis 223 is secured above the sun gear 217 which is provided at a center of the unit so as to be integral with the sun gear 217. An inner ring of a one way-clutch 224 is fitted on the output axis 223. A brake drum 225 is fitted on an outer ring of the one-way clutch 224. A brake belt 230 is wound around an outer periphery of the brake drum 225, one end of the brake belt 230 is secured to the housing 213, and the other end of the brake belt 230 is taken out via the outlet opening 214, and, as shown in FIG. 12, is connected to the inner shell 205 of the foot portion 202 via the wire 208. The housing 213 is secured to the circular holder 221 with a bolt 226.

When the thigh arm 211 and the leg arm 212 rotate relative to each other, the sun gear 217 rotates at the speed which is increased by eight times with the planetary gear mechanism. And the inner ring of the one-way clutch 224, which is integral with the sun gear 217, rotates, similarly. The outer ring of the one-way clutch 224 rotates integrally with the inner ring thereof in the direction in which the knee-ankle-foot orthosis flexes, while freely rotating in the direction in which the knee extends. The brake drum 225 is attached to the outer ring of the one-way clutch 224 integrally therewith to rotate together. Therefore, the brake drum 225 rotates at the speed which is increased by eight times of the rotation of the thigh arm 211 and the leg arm 212 only in the direction in which the knee-ankle-foot orthosis flexes. In order to stop the rotation of the brake drum 225, the brake belt 230 is wound therearound. One end of the brake belt 230 is secured to the housing 213, whereas, as shown in FIG. 12, the other end thereof is connected to the inner plastic part (inner shell) 205 via the wire 208. Consequently, when the foot portion 202 contacts the floor, and a load is applied thereto, the elastic body 207 is compressed to pull the wire 208, and accordingly pull the brake belt 230, thereby stopping the rotation of the brake drum 225 of which the speed is increased by eight times. As a result, the rotations of the thigh arm 211 and the leg arm 212 in the flexing direction thereof are stopped, thereby preventing giving way of the knee-ankle-foot orthosis. The rotations of the thigh arm 211 and the leg arm 212 in the extending direction thereof are made free by virtue of the one-way clutch 224, and consequently, the extension of the knee of the knee-ankle-foot orthosis is free. Therefore, when a load is applied to the foot portion 202, brakes are securely applied to prevent giving way of the knee-ankle-foot orthosis, whereas the extension of his knee is free regardless of the load of the foot portion 202. so as to be used with great convenience.

The gist of the present invention is that the giving way of the knee-ankle-foot orthosis is stopped with not a locking mechanism but a braking mechanism using a load of a patient's body weight.

### INDUSTRIAL APPLICABILITY

The present invention can be applied to the knee-ankle-foot orthosis.

1. A knee-ankle-foot orthosis with load brake comprising:
a knee axis gear which is secured to a thigh frame for taking a relative rotation of a knee axis to a leg frame;
a speed increasing gear train which is rotatably supported with said leg frame for increasing a rotation speed of said knee axis gear;
a braking section which is secured to a final stage of said speed increasing gear train for rotating integrally with a final stage gear;
a load detecting section which is provided in a foot holding portion for detecting a contacting pressure of a foot; and
braking means for connecting said load detecting section and said braking section to each other, and braking a rotation of said braking section when a load is applied to said load detecting section.

2. A knee-ankle-foot orthosis with load brake comprising:
a knee axis gear which is secured to a thigh frame for taking a relative rotation of a knee axis to a leg frame;
a speed increasing gear train which is rotatably supported with said leg frame for increasing a rotation speed of said knee axis gear;
a one-way clutch which is secured to a final stage of said speed increasing gear train for rotating integrally with a final stage gear, wherein:
said one-way clutch is arranged such that an input stage is integral with said final gear, whereas a braking section is provided in an output stage, that when said leg frame rotates in a flexing direction thereof, said one-way clutch becomes on to stop said rotation with said braking section, and that when said leg rotates in an extending direction thereof, said one-way clutch becomes off to make said rotation free;
a load detecting section which is provided in a foot holding portion for detecting a contacting pressure of a foot; and
braking means for connecting said load detecting section and said braking section to each other, and braking a rotation of said braking section when a load is applied to said load detecting section.

3. A knee-ankle-foot orthosis with load brake as claimed in one of claims 1 and 2, wherein:
said braking section is composed of a brake drum;
said load detecting section is composed of a first lever which moves upwardly due to a load of a heel, and a second lever which has a portion adapted to move downwardly due to the load after reversing a motion of said first lever; and
said braking means is composed of a wire, one end of which is secured to said second lever, and the other end of which is secured to said leg frame after being wound around said brake drum.

4. A knee-ankle-foot orthosis with load brake as claimed in one of claims 1 and 2, wherein:
said braking section is composed of one of a brake disc and a brake drum;
said load detecting section is composed of an oil hydraulic transducer which detects a load of a heel and varies an oil pressure due to said load; and
said braking means is composed of an oil hydraulic cylinder which is driven with said oil pressure of said oil hydraulic transducer, and one of a brake pad and a brake shoe, which is pressed with said oil hydraulic cylinder.

5. A knee-ankle-foot orthosis with load brake as claimed in one of claims 1 and 2, wherein:
said braking section is composed of one of a brake disc and a brake drum;
said load detecting section is composed of a load detector which detects a load of a heel and converts said load to an electric signal;
said braking means is composed of command value calculating means for calculating a brake torque command value required corresponding to said electric signal from said load detector, an actuator which is driven with an output of said command value calculating means, and one of a brake pad and a brake shoe, which is driven and pressed with said actuator.

6. A knee-ankle-foot orthosis with load brake comprising:
a planetary gear mechanism of which a first input axis is secured to a thigh frame, and a second input axis is secured to a leg frame for increasing a speed of a relative rotation of said first input axis and said second input axis, and transmitting said relative rotation to an output axis;
a braking section to which a rotation of said output axis of said planetary gear mechanism is transmitted;
a load detecting section which is provided in a foot holding portion for detecting a contacting pressure of a foot; and
braking means for connecting said load detecting section and said braking section to each other, and braking a rotation of said braking section when a load is applied to said load detecting section.

7. A knee-ankle-foot orthosis with load brake as claimed in claim 6, further comprising a one-way clutch which is interposed between said output axis of said planetary gear mechanism, and said braking section such that when the direction of a relative rotation of said thigh frame and said leg frame is the direction in which a knee flexes, said rotation is transmitted, and that when the direction of said relative rotation is the direction in which said knee extends, said rotation is not transmitted.

8. A knee-ankle-foot orthosis with load brake as claimed in one of claims 6 and 7, wherein:
said braking section is composed of a brake drum;
said load detecting section is composed of a foot portion which includes an inner shell on which a foot of a human body is adapted to be put, an outer shell which is adapted to contact a floor, and an elastic body which is interposed between said inner shell and said outer shell; and
said braking means is provided with a brake belt which is wound around said brake drum, one end of which is secured to a frame of said planetary gear mechanism, and the other end of which is connected to said inner shell of said foot portion.
